(19) United States
(12) Patent Application Publication
(10) Pub. No.: US 2013/0116574 A1
(43) Pub. Date: May 9, 2013

(54) METHOD FOR ASCERTAINING SPATIAL COORDINATES

(71) Applicants: Bruno Knobel, Laufen (CH); Charles Findeisen, Wettingen (CH)

(72) Inventors: Bruno Knobel, Laufen (CH); Charles Findeisen, Wettingen (CH)

(73) Assignee: NAVISWISS AG, Laufen (CH)

(21) Appl. No.: 13/809,962

(22) PCT Filed: Jul. 14, 2011
(86) PCT No.: PCT/IB11/02316

§ 371 Date: Jan. 14, 2013

(30) Foreign Application Priority Data
Jul. 15, 2010 (DE) 10 2010 027 336.8

(51) Int. Cl. A61B 5/11; A61B 5/107; A61B 5/00

(52) U.S. Cl. CPC A61B 5/1127; A61B 5/0077; A61B 5/1128; A61B 5/1121; A61B 5/1072; A61B 5/4851; A61B 5/4571
USPC 600/476

(57) ABSTRACT

The invention relates to a method for ascertaining spatial coordinates in which at least two markers are put on a living being and at least two cameras in a stereo arrangement are used to ascertain the spatial coordinates of the markers, wherein the spatial coordinates of the markers are compared with a reference and the difference is calculated and output. The invention also relates to quantitative length and angle measurements using optical, stereometric measurement systems for medical applications.

The invention relates to a method for ascertaining spatial coordinates, in which at least two markers are placed on a living being and at least two cameras in a stereo arrangement are used to ascertain the spatial coordinates of said markers. Furthermore, the invention relates to quantitative length and angle measurements using stereometric measurement systems for medical applications.

Today more than 1.3 million artificial hip joints (with a double-digit rate of growth) are being implanted in patients each year. The goal of this procedure is to restore the original or biometrically optimal leg length after implantation of the joint prosthesis. This goal is achievable, depending on the skill and expertise of the orthopedic surgeon and the patient's anatomy. For example, the orthopedic physician orients himself according to characteristic bone parts, for example, the femur and the pelvis, or specific locations on the foot joints.

In most procedures of this type, however, no suitable device is available for measuring leg lengths. Details of the procedure are thus based on the visual evaluation by the orthopedic surgeon.

A fundamental solution to this problem can be achieved by using the navigation systems available today. However, their use and handling are complex and are not very appropriate ergonomically. Furthermore, many operating rooms are not equipped with such systems.

Therapists and chiropractors record the condition of the patient and/or the effects of their treatment of the patient by using photographs and/or measurement means, for example, measurements of length or angle.

Users hereinafter are identified as being surgeons, orthopedic surgeons, chiropractors, therapists and others with appropriate medical knowledge. However, users may also be employees who perform the measurements for someone having medical training.

The object of the present invention is to provide a method with which measurements can be performed easily and rapidly on living creatures.

This object is achieved with a generic method in which the spatial coordinates of the markers are compared with a reference and the deviation is calculated and then output.

Advantageous variants of the embodiment are the subject matter of the dependent claims.

The invention begins with the basic idea that simple methods which can be handled with little effort are to be made available to the user with this measurement system. Thus the user can perform the measurement tasks supporting him quickly and reliably. The results of the measurements are used to evaluate the situation for the user, as an aid in performing the next operating steps or for the purpose of documentation.

A reference in this sense is either a data record of a previous measurement which describes a previous state or an ideal data record describing a result that is the goal or an intermediate state.

An optical stereometric measurement system (or just “measurement system”) is understood hereinafter to refer to the combination of a camera system and reference markers (just “markers”).

Such a measurement system is the state of the art. Additional properties of the measurement system are presented hereinafter to illustrate the basic idea of the method according to the invention.

The camera system consists of at least two stereometrically equipped cameras and means for analysis of the image data and for output of the results.

The camera system detects the markers, analyzes the image data by using known methods of camera image analysis, determines the spatial position of the markers in a coordinate system of the camera system by using known photogrammetric methods and makes available to the user the desired distances between the markers and/or angles between distances.

The camera system can be permanently integrated into the equipment of the treatment space. Alternatively, the camera system may be mounted on a mobile stand. The camera system may be equipped so that a coordinate axis of the camera coordinate system, for example, is parallel to the perpendicular. Thus, for example, the deviations of two markers from the horizontal can be measured quantitatively.

Alternatively, the camera system, which is not aligned, can be oriented with objects by means of known methods of camera image analysis. These objects may be inclined reference planes provided with markers, for example.

Another possibility is for the camera system to be mobile and operated manually.

The markers are provided by the user at or near biomechanically optimal and/or anatomically suitable locations on the patient or on objects. The markers may be, for example, small circles, x's or lines recorded using a felt-tip marker, directly above or between distinctive sections of bone. Another possibility for markers would be removable tattoos on the skin or adhesive markers, with or without the coding that is known from photogrammetry for identification of the markers.

Markers on the skin may be displaced in relation to the distinctive section of bone due to a treatment of the living creature or during said treatment. They may also be erased during the duration of the treatment. In such a case, the user may refresh or supplement the markers.

In practice, a measurement device of the type described in the introduction is used, so that the user applies markers to the patient at suitable locations and uses the measurement device to ascertain the spatial coordinates of the markers and their relationships to one another in the form of distances or angles.

The measurements may be used for a quantitative determination of a condition for a diagnosis. The measurements may also be used for quantitative determination of a condition before and after certain therapeutic or surgical procedures.

The results of the measurements may be used for the documentation by the user.

The general inventive idea consists of a measurement method for accompanying medical procedures and therapeutic measures using an optical camera system with at least two cameras in a stereo arrangement and at least two markers provided at suitable locations on a living creature, such that these markers are detected at least before and after a treatment of the living creature using the optical camera system, which determines the three-dimensional coordinates of these patterns in the camera coordinate system and makes them available to the user in a suitable form.

The method preferably includes the use of a fixedly installed camera system. According to another embodiment of the invention, mobile camera systems are used.

According to an especially advantageous embodiment, the method relates to a hip replacement surgery. Additional fields of application include spinal surgery, chiropractic or therapeutic treatments and measurement of the mobility of a body part.

The present invention is described in greater detail below as an example without restriction of the general idea of the invention, with reference to exemplary embodiments as illustrated in the drawings to which explicit reference is made hereinafter with respect to the disclosure of all the details according to the invention which are not explained in the text. In these drawings:

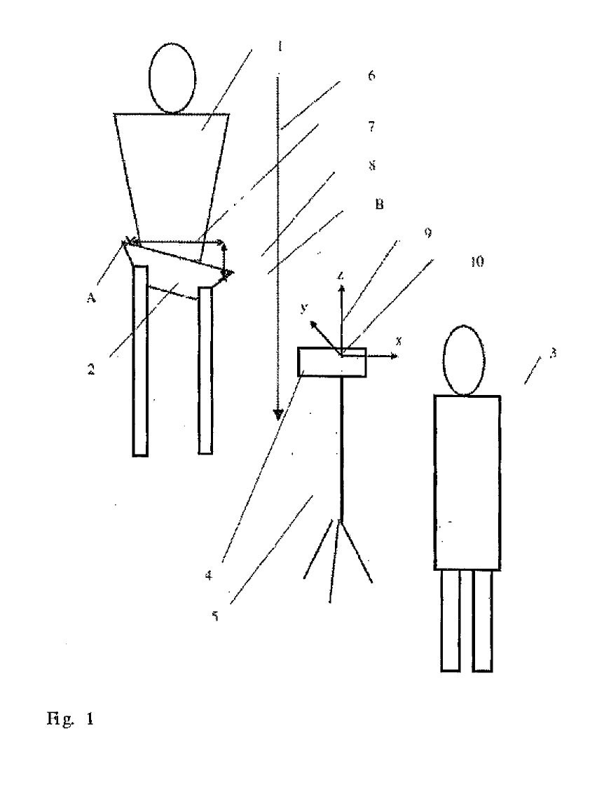

FIG. 1 shows schematically the measurement of the alignment of the pelvis with two markers and a camera system aligned with the perpendicular,

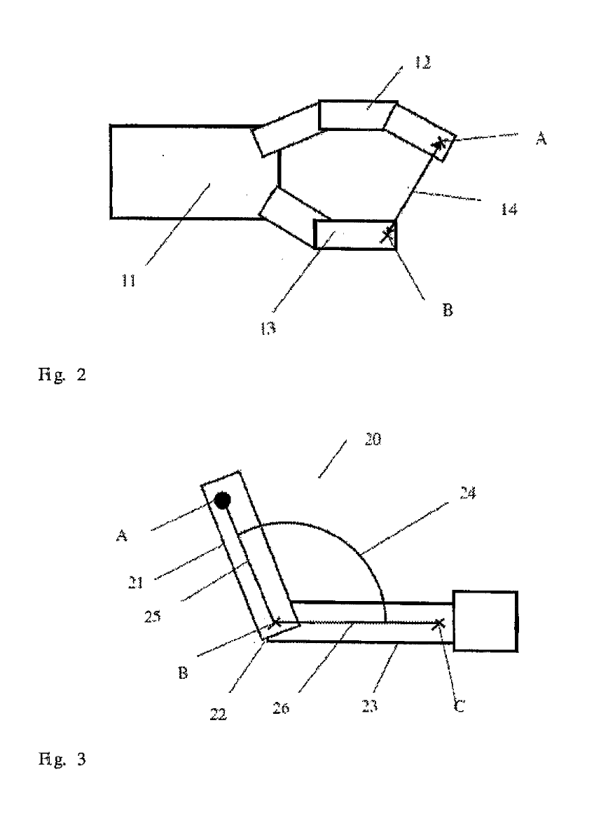

FIG. 2 shows schematically the measurement of the mobility of a hand using two markers,

FIG. 3 shows schematically the measurement of the mobility of an arm using three markers,

FIG. 4 shows schematically the measurement of the mobility of an arm using four markers,

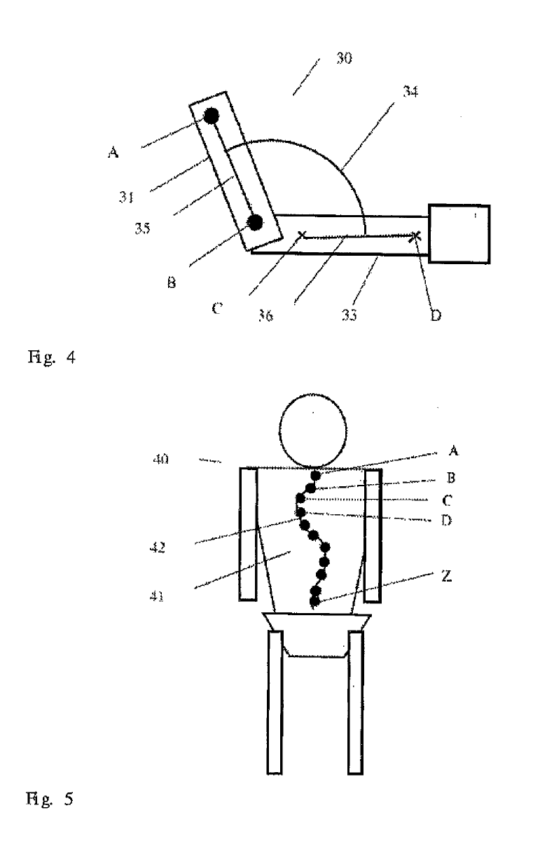

FIG. 5 shows schematically the measurement of the spine using several markers,

FIG. 6 shows schematically a patient, a camera system and four markers during hip replacement surgery,

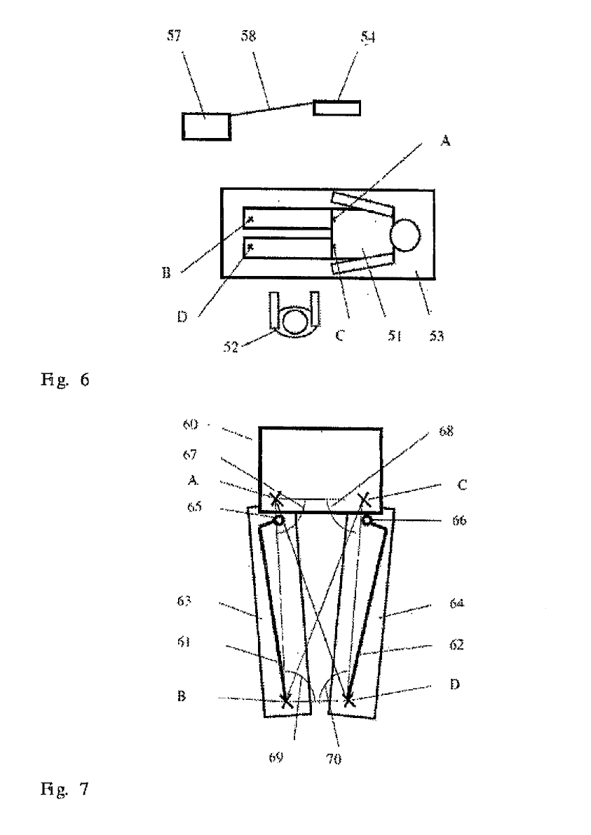

FIG. 7 shows schematically the area of the optimally aligned patient that is relevant in terms of the measurement technology, in a hip replacement surgery before the surgery,

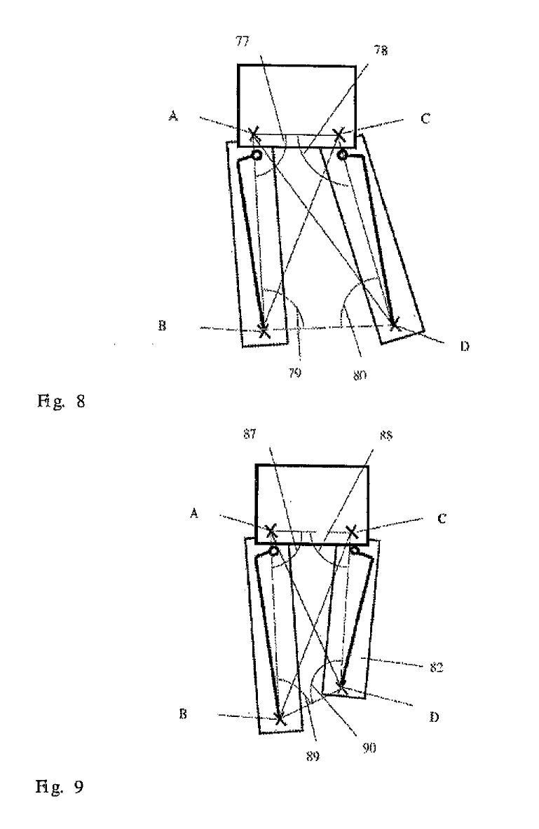

FIG. 8 shows schematically the area that is relevant in the measurement technology after performing certain steps on the patient, who is not optimally aligned after hip replacement surgery, and

FIG. 9 shows schematically the area that is relevant in the measurement technology after certain steps and the patient who is not aligned optimally after hip replacement surgery with the resulting shortening of the leg length.

FIG. 1 shows schematically the measurement of the orientation of the pelvis 2 of a patient 1 standing upright. The patient 1 has two markers A and B on his skin, these markers having been applied by the user 3 directly in the immediate vicinity of two specific pelvic bones. The measurement system 4 on the stand 5 is aligned with the patient 1, so that the z axis of the camera coordinate system 9, for example, is parallel to the perpendicular 6, and the zero point 10 is at approximately the same height as the two markers A and B.

The user 3 measures the spatial coordinates of the markers A and B. The measurement system thus calculates the horizontal distance 7 and the height difference 8 of the markers A and B. When the pelvis is aligned horizontally, the height difference 8 will be very small in comparison with the distance 7. The height difference 8 may amount to a few centimeters in the case of legs of unequal length, for example.

FIG. 2 shows schematically the mobility of a hand 11 with two markers A on the tip of the index finger 12 and B on the tip of the thumb 13. The measurement system, which is not shown in the drawing here, need not be specially aligned for this measurement because in this example only the distance 14 between the markers A and B is of interest for the user.

FIG. 3 shows schematically the measurement of the mobility of an arm 20 with the three markers A on the upper arm 21 as a circle, B on the elbow 22 and C on the forearm 23 as an “x.” The angle 24 between the line AB 25 and the line BC 26 serves as a measure of mobility. The measurement system, which is not shown here, need not be aligned with the perpendicular for this measurement.

FIG. 4 shows schematically the measurement of the mobility of an arm 30 with four markers, A and B as circles on the upper arm 31 and C and D as x's on the forearm 33. The angle 34 between the two lines AB 35 and CD 36 serves as a measure of mobility. The measuring system, which is not shown here, need not be aligned with the perpendicular for this measurement, for example.

FIG. 5 shows schematically the measurement of the shape of the spine 42 of a patent 40 standing upright with several markers A, B, C, D, Z, which are applied to the skin on the back 41 directly above the spinal processes, for example. The measurement system not shown here may be aligned with respect to the perpendicular, for example, for this measurement. The coordinates of the measured markers are made available to the user in processed form.

FIG. 6 shows schematically the patient 51, the measurement system 54, 57, 58 with four markers A, B, C, D in hip replacement surgery. The patient 51 on the surgical table 53 is oriented in an optimal orientation for the user 52. In this measurement, the camera system 54 is above the markers A, B, C and D. This camera system 54 is fixedly mounted above the surgical table 53. The images recorded with the camera system 54 are transmitted to the module 57 with the computation unit and display screen via the communication link 58, and the results are made available to the user 52.

FIG. 7 shows a schematic diagram of the relevant areas of the surgical arrangement. Before the procedure, the four markers A, B, C and D are placed by the user in the anatomically and/or biomechanically correct locations for the user on the skin of the patient 60 by using a felt-tip marker. These locations can be selected by the user, so that the method according to the invention supports his customary method of proceeding. The two points A and C are located on the skin above the protruding pelvic bone (anterior superior iliac spine). The two points B and 0 are marked on areas of skin close to suitable parts of the joints of the foot. The points 65 and 66 are the focal points of the hip joints. The pivot point 65 is typically located close to the line AB. The pivot point 66 is typically located close to the line CD. The right and left leg bones (femur, tibia, fibula) 61 and 62 as well as the right leg 63 and the left leg 64 of the patient are shown schematically.

A mirror symmetry that is useful and helpful in analysis of the measurement results from the positions of the markers and from a biomechanical alignment of the patient that is optimal for the user.

The following relationships between lines and angles can thus be determined: all four markers A, B, C and D are typically approximately at the same level. The line BD is somewhat shorter than the line AC if both feet are close together. The lines AC and BD are approximately parallel to one another. The diagonal lines AD and BC are of approximately the same length. The angle 67 between the lines AB and AC is approximately equal to the angle 68 between the lines AB and BD. The angle 69 between the lines AB and BD is approximately equal to the angle 70 between the lines CD and BD. For this example, the lines AB and CD are defined as leg lengths.

Thus the six lines AB, AC, AD, CD, CB and BD and the four angles 67, 68, 69, 70 determined in the first measurement and are shown in FIG. 7 are known for the starting position.

A change in leg length resulting from the procedure can be described mathematically by comparing the second measurement performed after the procedure with the first measurement using the six lines AB, AC, AD, CD, CB and BD and the four angles 67, 68, 69, 70.

For example, if surgery is performed on the left hip, then the length of the vector CD may change with respect to the first measurement. This change in leg length is essentially the difference between the lines CD before the procedure and the lines CD after the procedure. In addition, the two diagonals AD and CB and the two angles 67 and 68 show how the patient was oriented in the measurements on the surgical table. The patient need not be in the same location in space and in the same location with respect to the camera system as in the first measurement. The goal in the measurement after the procedure is for the patient to assume approximately the same optimal biomechanical alignment as in the first measurement. Then the change in leg length due to the procedure can be calculated. Either deviations in the biomechanical patient alignment in the second measurement in comparison with the first measurement are detected by this analysis and are taken into account in calculation of the change in leg length.

FIG. 8 shows schematically the position of the markers in a patient who is not aligned optimally after the procedure without any change in the length of the left leg. Suboptimal alignment of the patient is manifested, for example, by the fact that the diagonal lines AD and BC are not equal in length, the length of the line BD has changed and the angles 77, 78, 79, 80 have changed. The user can optimally align the patient for the measurement and evaluation of the quality of the procedure with the help of this information.

FIG. 9 shows schematically the position of the markers in a patient who is optimally aligned but with shortening of the length of the leg 82 caused by the procedure. A suboptimal result of the procedure is manifested by the fact that the diagonal lines AD and BC are not the same, the length of the line BD is altered and the angles 88, 89 and 90 have changed. The user can adjust the remaining course of the procedure to this situation with the help of this information and can make the required corrections.

At the end of the procedure, the patient can be measured one last time. For the user, this measurement serves as quality control for the procedure.

The detected images and analysis records compiled can be archived for future applications.

This invention is not suitable just for use in hip replacement surgeries but in all cases when a biomechanical change is possible as a result of a medical treatment, it can be documented with measurement technology using the method described here, and the results of the analyses may be made available to the user.

The measurement includes a stereo photograph or a series of stereo photographs in which the user is treating, moving or shifting the patient in the desired manner or the patient must move certain body parts.

For the surgeon, a large area of application is on the spine, chiropractic treatment, checking and measuring the mobility of a body part such as the spine, a joint of the hand, finger, foot or shoulder.

Another application of the invention is to monitor and/or modify the follow-up treatment during the course of healing, for example.

1. A method for determining spatial coordinates, in which at least two markers are made on a living creature and the spatial coordinates of these markers are determined using at least two cameras in a stereo arrangement, wherein the spatial coordinates of the markers are compared with a reference and the deviation is calculated and output.

2. The method according to claim 1, wherein the spatial coordinates are determined at least before and after a change in the distance between the markers caused by a treatment of the living creature.

3. The method according to claim 2, wherein the living being is positioned differently in space before the treatment in comparison with after the treatment.

4. The method according to Claim 1, wherein additional markers are made on the living creature during the treatment.

5. The method according to claim 1, wherein the change in the distance between two markers is calculated from the spatial coordinates.

6. The method according to claim 1, wherein the change in the angle between the three markers is calculated from the spatial coordinates.

7. The method according to claim 1, wherein the markers are made on the skin of the living creature.

8. The method according to claim 1, wherein the markers are made above significant bone points of the living creature.

9. The method according to claim 1, wherein a change in a length or a change in an angle is determined by comparison with a reference.

10. The method according to claim 1, wherein an absolute length is determined by comparison with a previously known measure detected by the cameras.

11. The method according to claim 1, wherein the cameras are arranged in stationary positions in space.

12. The method according to claim 1, wherein the cameras are arranged so they are fixedly connected to one another but are mobile in space.
